(19) United States
(12) Patent Application Publication
(10) Pub. No.: US 2008/0161557 A1
(43) Pub. Date: Jul. 3, 2008

(54) Synthesis of 2-Methyl-4-(4-Methyl-1-Piperazinly)-10H-Thieno(2,3-B) (1,5) Benzodiazepine and Salts Thereof

(75) Inventors: Tomaz Mesar, Trzin (SI); Anton Copar, Smartno pri Litiji (SI); Hubert Sturm, Innsbruck (AT); Johannes Ludescher, Breitenbach (AT)

Correspondence Address: SANDOZ INC, 506 CARNEFIE CENTER, PRINCETON, NJ, 08540, US

(73) Assignee: LEK PHARMACEUTICALS D.D., LJUBLJANA (SL)

(21) Appl. No.: 10/598,816

(22) PCT Filed: Mar. 17, 2005
(86) PCT No.: PCT/EP2005/002876

§ 371 Date: Dec. 14, 2006

(30) Foreign Application Priority Data
Mar. 18, 2004 (SL) P200400079
Nov. 16, 2004 (SI) P200400311

(51) Int. Cl. C07D 513/04

(52) U.S. Cl. 540/557

(57) ABSTRACT

The invention belongs to the field of organic chemistry and relates to a new process for the purification of olanzapine comprising preparation of acid addition salts of olanzapine and transformation thereof into a pharmaceutically acceptable pure and discoloured final product. The present invention also relates to new processes for the preparation of pure olanzapine.

### FIELD OF THE INVENTION

The invention belongs to the field of organic chemistry and relates to a new process for the purification of 2-methyl-4-(4-methyl-1-piperazinyl)-10H-thieno[2,3-b][1,5]benzodiazepine (olanzapine) comprising preparation of acid addition salts of olanzapine and transformation thereof into a pharmaceutically acceptable pure and discoloured final product. The present invention also relates to processes for the preparation of pure olanzapine.

### BACKGROUND OF THE INVENTION

Olanzapine is a pharmaceutical active substance from the group of antipsychotics, applicable for the treatment of different mental diseases and conditions such as, for example, disorders of the central nervous system, schizophrenia, hallucination, acute mania, depression, and the like.

Chemically, it belongs to the group of the benzodiazepines and is 2-methyl-4-(4-methyl-1-piperazinyl)-10H-thieno[2,3-b][1,5]benzodiazepine (formula 1).

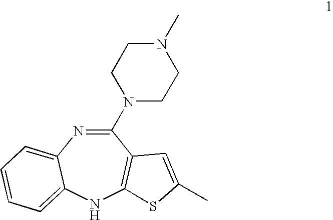

Olanzapine and analogues thereof are encompassed for the first time within a general formula in patent GB 1,533,235. Even if this reference discloses a general alkylation for the synthesis of 4-substituted derivatives, methylation as such was not explicitly mentioned. The alkylation reactions were carried out in ethanol using triethylamine as a base and R′-Cl as an alkylating agent wherein R′ represented different radicals.

In GB 1,533,235 two routes of synthesis are disclosed with general formulas, being described in detail in the basic patent, EP454436B1. EP454436B1 discloses two different one-step processes for olanzapine preparation. The first described process is a reaction of 4-amino-2-methyl-10H-thieno[2,3-b][1,5]benzodiazepine hydrochloride with N-methylpiperazine in an organic solvent such as anisole, toluene, dimethylformamide or dimethyl sulfoxide, preferably at a temperature from 100 to 150° C. to yield olanzapine (Scheme 1).

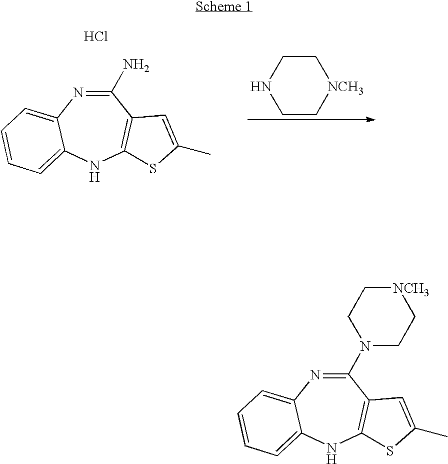

The second process disclosed in EP 454436 B1is the reaction of N-methylpiperazine with methyl-2-(2-aminoanilino)-5-methylthiophene-3-carboxylate in the presence of titanium tetrachloride (Scheme 2).

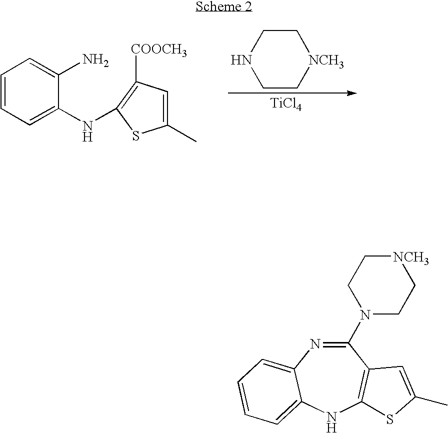

The same patent also mentions the formation of acid addition salts of olanzapine and their potential use as intermediates in olanzapine purification process and in pharmaceutical use. However none of these acid addition salts were prepared or characterized and no process or experiment using any of said acid addition salt was disclosed.

A major problem associated with both synthesis is the colouration of the final product, due to the use of thiophenic chemistry.

As disclosed in EP454436B1 and US equivalent U.S. Pat. No. 5,229,382, olanzapine obtained according to the first synthesis (Scheme 1) is then purified by recrystallization from acetonitrile, whereas olanzapine prepared according to the second route (Scheme 2) is further purified by column chromatography on Florisil and recrystallized from acetonitrile.

The problem of colouration of olanzapine is well known and has already been reported in EP733635B1 that refers to U.S. Pat. No. 5,229,382. Both references have shown that olanzapine may still contain traces of undesirable colour even after purification with activated charcoal. In EP733635B1 the inventors have tried to solve the colouration problem by preparing a new crystal form of olanzapine.

Other synthesis processes for the preparation of olanzapine have been described in the prior art. For example patent application WO 04/000847 discloses a two step synthesis from 4-amino-2-methyl-10H-thieno[2,3-b][1,5]benzodiazepine hydrochloride via 2-methyl-4-(1-piperazinyl)-10H-thieno[2,3-b][1,5]benzodiazepine (i.e. N-desmethylolanzapine) with reductive N-methylation (using formaldehyde and metal boron hydride). WO 04/000847 describes that the methylation process is carried out in methanol. Disadvantages of processes disclosed in WO 04/000847 are low yields and bad quality of the final product.

Reaction of 4-amino-2-methyl-10H-thieno[2,3-b][1,5]benzodiazepine hydrochloride with piperazine to produce N-desmethylolanzapine (Scheme 3) was published in Bioorganic & Medicinal Chemistry Letters, Vol. 7, No. 1, pp. 25-30, 1997. A mixture of dimethyl sulfoxide/toluene=¼ was used as a solvent mixture.

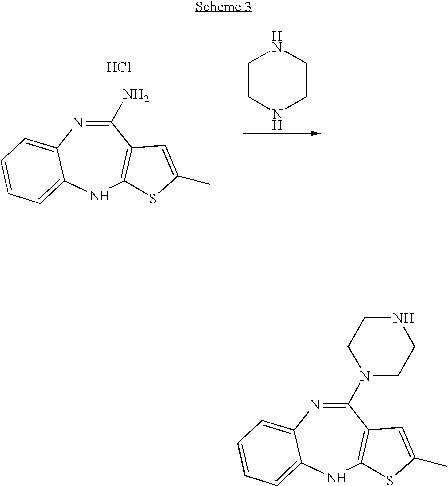

WO04/089313 discloses olanzapine acid salts, solvates and co-crystals and their use as active pharmaceutical ingredient in formulation. The preparation of fumaric, maleic and malonic acid addition salts of olanzapine is disclosed in WO 04/089313. Olanzapine acid addition salts disclosed in this application exhibit specific aqueous solubility from 50 μg/ml to 100 mg/ml.

It is well known to a skilled person that most chemical reactions are not completely finished, may be reversible or are driven simultaneously with some other parallel reactions. Starting materials or side reaction products are usually found as impurities in the isolated main product which should therefore be further purified. The simplest way of purification includes various recrystallization and precipitation procedures which are usually less effective if the impurities have physico-chemical properties very similar to the main product.

In the case olanzapine is prepared according to the one step processes disclosed in EP 454436 B1, the starting material, 4-amino-2-methyl-10H-thieno[2,3-b][1,5]-benzodiazepine, is found as an impurity in the final product olanzapine.

Disadvantage of the reaction published in said review Bioorganic & Medicinal Chemistry Letters, Vol. 7, No. 1, pp. 25-30, 199 is a dark coloured product.

In the case of preparation of olanzapine by a two-step process, as disclosed also in patent application WO 04/000847 the presence of 4-amino-2-methyl-10H-thieno[2,3-b][1,5]-benzodiazepine hydrochloride is not critical but various other similar compounds could be found as impurities, such as 4-(4-formylpiperazinyl)-2-methyl-10H-thieno[2,3-b][1,5]-benzodiazepine and N-desmethylolanzapine. For all these impurities that have a thienobenzodiazepine ring system as a part of the molecule skeleton and because it represents a great part of molecule, said ring system is crucial for similarity of physico-chemical properties of said impurities compared to olanzapine.

It has now been found that olanzapine cannot be efficiently separated from its highly related impurities using repeated crystallisation of crude olanzapine.

It would be therefore desirable to develop alternative processes for the preparation of a pharmaceutically acceptable pure and discoloured olanzapine.

### SUMMARY OF THE INVENTION

We have now discovered a new and straightforward process for the purification of olanzapine comprising the transformation of olanzapine to an acid addition salt thereof, a separation step and then recovering of olanzapine from said addition salt.

In the first embodiment the invention concerns a process for the purification of olanzapine characterised in that said process comprises the following steps:
a) mixing olanzapine with an organic acid in an organic solvent or a mixture of organic solvents to form an olanzapine acid addition salt,
b) precipitating and isolating the olanzapine acid addition salt and
c) transformation of the olanzapine acid addition salt to olanzapine.

In another embodiment, the invention concerns a process for the preparation of N-desmethylolanzapine comprising reacting 4-amino-2-methyl-10H-thieno[2,3-b][1,5]benzodiazepine hydrochloride and piperazine in a solvent or in a mixture of solvents comprising at least one aliphatic alcohol having a higher boiling point.

In another embodiment, the invention concerns a process for the synthesis of 2-methyl-4-(4-methyl-1-piperazinyl)-10H-thieno[2,3-b][1,5]benzodiazepine (olanzapine) of a light colour without dark brown or green tinges comprising the N-methylation of N-desmethylolanzapine with a methylating agent, optionally in the present of a strong base in an organic solvent or in the mixture of organic solvents.

In another embodiment, the invention concerns a process for the preparation of olanzapine in the form of an acid addition salt comprising
a) mixing olanzapine with an organic acid in a solvent or a mixture of solvents and,
b) precipitating and isolating the olanzapine acid addition salt by separation of crystals.

In another embodiment, the invention concerns a process for the preparation of olanzapine in the form of an acid addition salt comprising the following steps: 4-amino-2-methyl-10H-thieno[2,3-b][1,5]benzodiazepine hydrochloride is reacted with N-methylpiperazine to yield olanzapine and obtained olanzapine is transformed to an acid addition salt thereof.

In another embodiment, the invention concerns olanzapine in a form of an acid addition salt wherein said acid is selected from the group consisting of benzoic acid and sulfonic acids.

In another embodiment, the invention concerns a process for the preparation of olanzapine from an acid addition salt thereof by recovering olanzapine from the said acid addition salt.

In another embodiment, the invention concerns a process for the preparation of olanzapine crystal form I from an acid addition salt of olanzapine wherein the crystals are isolated from an organic solvent.

In another embodiment, the invention concerns a process for the preparation of olanzapine crystal form II from an acid addition salt of olanzapine wherein the crystals are isolated from an organic solvent.

In another embodiment, the invention concerns the use of organic acids in the process of the preparation of olanzapine wherein olanzapine is purified via the formation of an acid addition salt thereof.

In another embodiment, the invention concerns a process for the preparation of a derivative of N-desmethylolanzapine of formula 2, wherein R means an organic radical such as acetyl, propionyl, chloroacetyl and the like, comprising reacting N-desmethylolanzapine with an organic acid or substituted organic acid or organic acid derivative of formula RX or with an organic acid anhydride. Said RX corresponds to organic acid derivative, particularly preferred is organic acid halide, such as acetyl halide, propionyl halide, chloroacethyl halide and the like, where X is selected from a group of Cl, Br or I, particularly preferred is Cl. Organic acid anhydride used can be acetic anhydride, propionic anhydride, phthalic anhydride and the like.

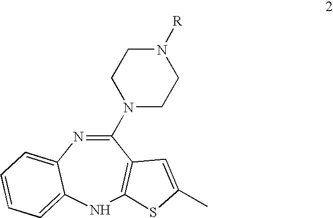

In another embodiment, the invention concerns olanzapine prepared from N-desmethylolanzapine by methylation process that yields N-desmethylolanzapine content in the final product of olanzapine in less than 0.1%.

In another embodiment, the invention concerns a process for the preparation of olanzapine in the form of an acid addition salt comprising the following steps:
a) N-desmethylolanzapine is reacted with a methylating agent to yield olanzapine,
b) the obtained reaction mixture is diluted with water and acidified with an acid,
c) to the reaction mixture, an organic solvent is added and the phases are separated,
d) the obtained water phase is neutralized and olanzapine is extracted with an organic solvent to obtain the organic solvent phase and,
e) an organic acid or substituted organic acid or organic acid derivative of previously defined formula RX; wherein R represents an organic radical such as acetyl, propionyl, chloroacetyl and X is selected from a group of Cl, Br or I, particularly preferred is Cl; or an organic acid anhydride as previously defined, is added to the organic phase to form a N-substituted N-desmethylolanzapine derivative of formula 2

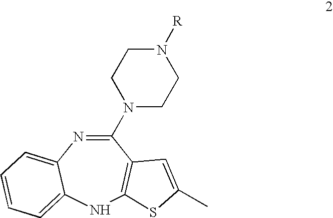

f) the obtained organic solvent phase is optionally evaporated and the residue is diluted with a second organic solvent,
g) an organic acid is added either to the obtained diluted solution or directly to the olanzapine extract from said extraction in step d) and,
h) precipitated olanzapine acid addition salt is isolated by separation of the crystals.

In another embodiment, the invention concerns olanzapine prepared from N-desmethylolanzapine by an N-methylation process, that contains less than 0.05% of piperazine 1,4-bis-4-yl-(2-methyl)-10H-thieno-[2,3-b][1,5]benzodiazepine.

In another embodiment, the invention concerns a process for the preparation of olanzapine comprising the following steps:
a) transformation of 4-amino-2-methyl-10H-thieno[2,3-b][1,5]-benzodiazepine hydrochloride to 2-methyl-4-(1-piperazinyl)-10H-thieno-[2,3-b][1,5]benzodiazepine,
b) transformation of 2-methyl-4-(1-piperazinyl)-10H-thieno[2,3-b][1,5]-benzodiazepine to crude olanzapine,
c) transformation of crude olanzapine to an acid addition salt thereof and
d) transformation of an acid addition salt of olanzapine to olanzapine.

In another embodiment, the invention concerns a pharmaceutical composition comprising 2-methyl-4-(4-methyl-1-piperazinyl)-10H-thieno[2,3-b][1,5]benzodiazepine (olanzapine) whereby olanzapine is prepared from an acid addition salt thereof.

In another embodiment, the invention concerns a use of olanzapine prepared according to one of the processes disclosed in this invention, for the preparation of amedicament for the treatment of different mental diseases and conditions.

In another embodiment, the invention concerns a pharmaceutical formulation comprising at least one pharmaceutically acceptable ingredient and olanzapine prepared according to one of the processes disclosed in this invention.

In another embodiment, the invention concerns the use of olanzapine prepared according to one the processes disclosed in this invention for the preparation of the pharmaceutical formulation together with at least one pharmaceutically acceptable ingredient.

### DETAILED DESCRIPTION OF THE INVENTION

The present invention provides a new process for the purification of olanzapine comprising the transformation of olanzapine to an acid addition salt thereof, a separation step and the recovering of olanzapine from said addition salt.

It is known in the state of the art that formation of acid addition salts of a substance and crystallization thereof from a solution can successfully purify said substance from impurities which cannot form acid addition salts and also from impurities which can form such salts but the properties thereof differ in a great extent from the said substance. It was found that olanzapine contaminated with highly related impurities can effectively be purified by transformation thereof into an acid addition salt which can be precipitated from solvents with an excellent purifying capacity. This is in contrast to olanzapine itself and some other olanzapine salts, particularly inorganic salts. We found that suitable organic acids that could be used for the preparation of olanzapine acid addition salts having capability for separation are carboxylic acids with at least one carboxylic group, such as oxalic, fumaric and benzoic acid, preferably oxalic acid. Sulfonic acids could also be used.

The purification process of olanzapine according to the present invention comprises the following steps:
a) mixing olanzapine with an organic acid in an organic solvent, or a mixture of organic solvents to form an olanzapine acid addition salt,
b) precipitating and isolating the olanzapine acid addition salt and,
c) transformation of the olanzapine acid addition salt to olanzapine.

Preferred organic acid in step a) are selected from the group consisting of sulfonic acids or carboxylic acid. Preferred carboxylic acid are selected from the group consisting of oxalic acid, fumaric acid and benzoic acid.

Preferred organic solvent in step a) are selected from the group consisting of tetrahydrofurane, acetone, dimethylformamide and acetonitrile.

Preferred mixture of organic solvents in step a) is a mixture of tetrahydrofurane with at least one polar solvent. Preferred polar solvent are selected from the group consisting of dimethylformamide, dimethylacetamide, N-methylpyrrolidone, 1,3-dimethyl-3,4,5,6-tetrahydro-2(1H)-pyrimidinone, 1,3-dimethyl-2-imidazolidinone, tetramethylurea, dimethyl sulfoxide, sulfolane, acetone and acetonitrile.

Preferred purification process according to the invention comprises the following substeps in step (c):
i) dissolving an acid addition salt of olanzapine in water,
ii) adjusting pH of the obtained solution to about 8-10,
iii) extracting olanzapine from the water phase to organic solvent phase and,
iv) isolating the acid addition salt of olanzapine from organic solvent phase by concentrating the solution and separation of the crystals.

Another embodiment of the present invention is a process for the preparation of olanzapine in the form of an acid addition salt, characterized in that said process comprises the steps of:
a) mixing olanzapine with an organic acid, preferably selected from the group consisting of benzoic acid and sulfonic acids in a solvent or a mixture of solvents and
b) precipitating and isolating the olanzapine acid addition salt by separation of crystals.

Preferably, said organic solvent, mixture of organic solvents and polar solvent correspond respectively to the same as previously described.

Another embodiment of the present invention is a process for the preparation of olanzapine, preferably in a crystalline form, characterized in that it is prepared from olanzapine acid addition salt by recovering from the said salt.

Preferably, said recovering step comprises the substeps of step (c) as previously described.

Suitable acid organic compounds which are used in this purification step are commercially available. On the other hand olanzapine as starting product can be synthesized according to the synthesis hitherto disclosed in the prior art, for example in EP454436B1; U.S. Pat. No. 5,229,382; EP733635B1 or WO04/089313.

We have now discovered alternative processes for the preparation of olanzapine which can optionally be purified according to the invention as previously disclosed.

Accordingly, the present invention further provides a new process for the synthesis of pure and discoloured olanzapine from 4-amino-2-methyl-10H-thieno[2,3-b][1,5]benzodiazepine hydrochloride via N-desmethylolanzapine as intermediate. The obtained crude olanzapine may then optionally enter into a purification process where the olanzapine acid addition salt is formed in a first step, followed by isolation thereof. Thereby the impurities from the crude olanzapine preparation process remain in the solution. In the further step of the purification olanzapine acid additional salt is easily transformed to the pure and pharmaceutically acceptable olanzapine without a dark colour tinge.

Said purification process via transformation of olanzapine to an acid addition salt thereof can also be used for olanzapine formed by one step process wherein 4-amino-2-methyl-10H-thieno[2,3-b][1,5]benzodiazepine reacts with N-methylpiperazine and yields crude olanzapine which directly enter into the purification process. Purified olanzapine from the invented procedure could be finally prepared in various crystal forms, such as form I or form II.

Moreover, it has been found that when an acid addition salt of olanzapine is prepared from a crude olanzapine deriving from a two step synthesis via N-desmethylolanzapine intermediate, N-desmethylolanzapine could precipitate from solvents in the form of an acid addition salt and remains in the final product as a contaminant. However it has very surprisingly been found that derivatives of N-desmethylolanzapine, such as acetyl, do not precipitate from organic solvents as an acid addition salt and remain in the mixture after the formation of crude olanzapine acid addition salt. In such a way N-desmethylolanzapine could be separated from olanzapine. Said method of purification can be very effective to ensure the level of any single impurity of pharmaceutical grade olanzapine below 0.1% and the method can be particularly important for removing said N-desmethylolanzapine which can otherwise be very difficult for separation from olanzapine. The level of impurities decreased in an appreciable extent even if the level of impurities in crude olanzapine was high.

Additionally, we have found out that during the first step of the olanzapine two step synthesis process (i.e. reaction of 4-amino-2-methyl-10H-thieno[2,3-b][1,5]-benzodiazepine with piperazine), a by-product is formed, identified as a dimer of the starting compound where two molecules of 4-amino-2-methyl-10H-thieno[2,3-b][1,5]-benzodiazepine hydrochloride are connected to nitrogen atoms of piperazine. The chemical name of this by-product is piperazine 1,4-bis-4-yl-(2-methyl)-10H-thieno-[2,3-b][1,5]benzodiazepine (scheme 4).

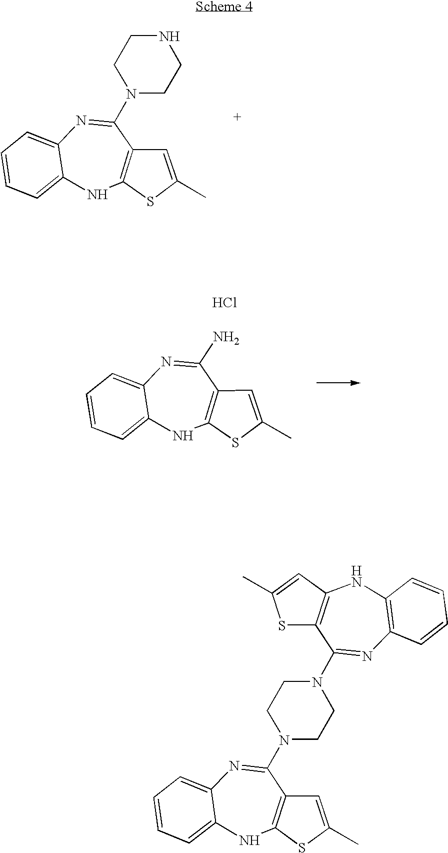

HPLC analysis showed that said dimer impurity occurred in the range of about 1-4% compared to the resulting N-desmethylolanzapine. We also found that molar surplus of starting piperazine as to 4-amino-2-methyl-10H-thieno[2,3-b][1,5]benzodiazepine hydrochloride in the reaction mixture yields less said dimer.

In the two-step synthesis of olanzapine according to the present invention 4-amino-2-methyl-10H-thieno[2,3-b][1,5]benzodiazepine hydrochloride is reacted with piperazine to form N-desmethylolanzapine. Olanzapine is then obtained via the methylation of said N-desmethylolanzapine (scheme 5):

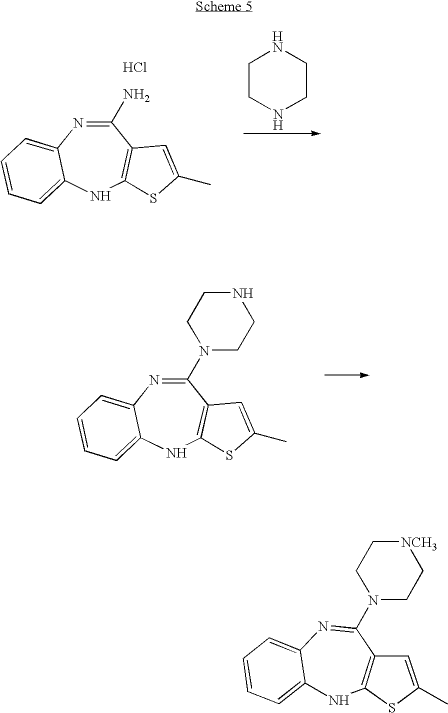

It has surprisingly been found that olanzapine with a desired bright yellow colour can be obtained from a dark brown or green coloured starting product (i.e: 4-amino-2-methyl-10H-thieno[2,3-b][1,5]benzodiazepine hydrochloride) if olanzapine is prepared by two-step synthesis according to the present invention via the isolation of N-desmethylolanzapine.

EP454436B1 already discloses the synthesis of olanzapine consisting in reacting 4-amino-2-methyl-10H-thieno[2,3-b][1,5]benzodiazepine hydrochloride with an excess of N-methylpiperazine in a 1:4 mixture of DMSO and toluene, which is the same type of the reaction (i.e. substitution of an amino group with N-methylpiperazine) as here disclosed in the first step (i.e. substitution of an amino group with piperazine) (scheme 4).

We have surprisingly found that for both the reaction rate and the reaction yield for said type of reaction, it is better to use at least one branched or unbranched aliphatic alcohol having a higher boiling point. According to the present invention a higher boiling point means a boiling temperature that is preferably above 100° C., more preferably above 115° C. Preferred aliphatic alcohol with a higher boiling point is n-butanol. As an alternative, a mixture of solvents containing at least one branched or unbranched aliphatic alcohol having a higher boiling point and at least one non alcoholic solvent having a higher boiling point, preferably n-butanol can be used. Preferred non alcoholic solvent having a higher boiling point is aromatic hydrocarbon solvent, especially xylene, toluene, ethylbenzene, anisole or the like: Suitable mixture are for example, a mixture of n-butanol and xylene or n-butanol and toluene in ratios n-butanol/aromatic hydrocarbon=30/70 to 100/0, preferably in ratios between 40/60 and 70/30.

Instead of having an excess of piperazine, additional inorganic or organic bases may be added to the reaction mixture. Preferred bases are tertiary amines, such as, for example, triethylamine, ethyldiisopropylamine or diazabicyclooctane.

When the reaction is carried out in a solvent system comprising n-butanol, N-desmethylolanzapine precipitates after warm water is added to the reaction mixture. Obtained N-desmethylolanzapine has already essentially lost dark brown or green colour. It is further possible to get rid of the remaining colour by washing N-desmethylolanzapine with an organic solvent, such as esters, e.g., ethyl acetate, isopropyl acetate, butyl acetate and the like. Warm water has a temperature between about 25 and about 70° C., preferably between about 30 and about 50° C. By using warm water, a sticky lumpy precipitate dissolves into smaller particles. Thereby a better filterability and a better quality (related to the colour) of the product can be achieved.

The second step of the synthesis corresponds to the N-methylation of the piperazine group of N-desmethylolanzapine (see scheme 6) to form crude olanzapine. For the methylation, different methylating agents can be used, for example, dimethyl sulfate or methyl sulfonates; such as methyl toluenesulfonate, methyl methanesulfonate, methyl trifluoromethanesulfonate; or methyl halogenides, preferably methyl iodide. The reaction can be carried out in different organic solvents, such as ethers or cyclic ethers, e.g. tetrahydrofuran; ketones, e.g. acetone; amides, e.g. dimethylformamide; nitriles, e.g. acetonitrile; or alcohols or mixtures of said solvents with other solvents, preferred is a mixture of tetrahydrofurane with polar solvents. Such polar solvents are amides, such as dimethylformamide, dimethylacetamide and N-methylpyrrolidone; ureas, such as 1,3-dimethyl-3,4,5,6-tetrahydro-2(1H)-pyrimidinone, 1,3-dimethyl-2-imidazolidinone and tetramethylurea; and other solvents, such as dimethyl sulfoxide, sulfolane, acetone, acetonitrile and the like. Such mixtures of tetrahydrofurane and polar solvents are superior in ensuring a higher ratio of olanzapine in the product versus non-methylated (i.e. N-desmethylolanzapine) and dimethylated products like N,N-dimethylolanzapine (Scheme 6).

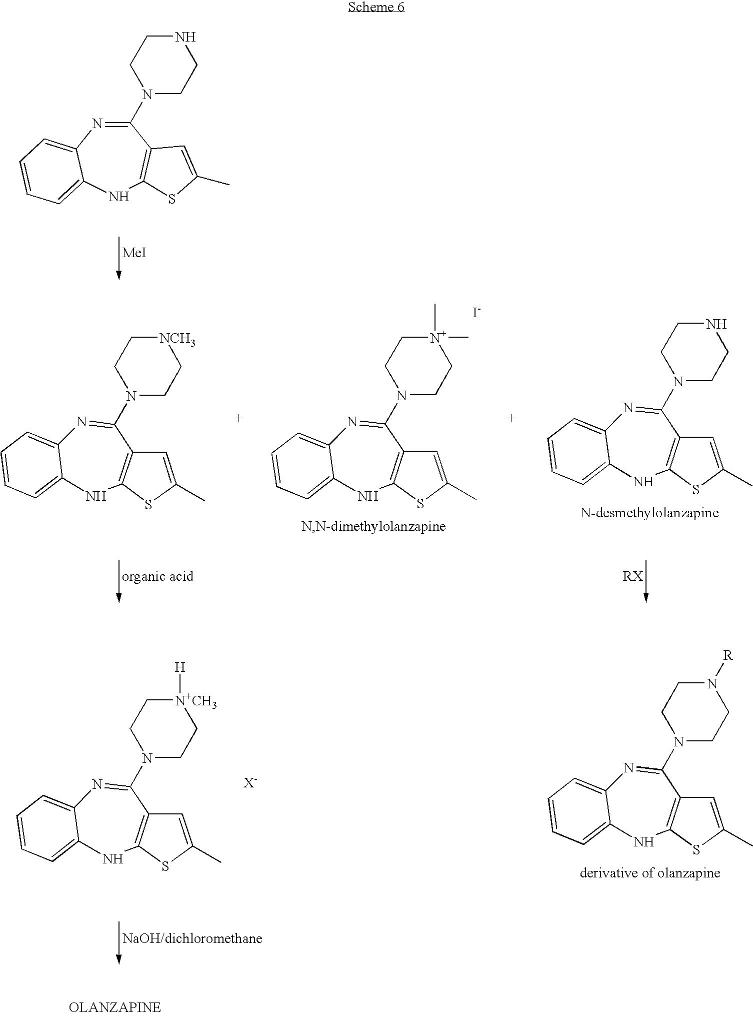

For said methylation reaction, alkaline conditions are preferred. Different amines may be used, such as triethylamine, diisopropylamine, dicyclohexylamine, ethyldiisopropylamine and diazabicyclooctane, or strong bases of alkaline or alkaline-earth metals, such as hydroxides, hydrides or alcoholates, for example sodium hydride, calcium hydride, potassium t-butoxide, sodium or potassium hydroxide as well as other inorganic bases, such as potassium or sodium carbonate. After the methylation of N-desmethylolanzapine under said conditions, olanzapine of a bright yellow colour without any brown or green tinge is obtained which does not require a subsequent removal of colour.

As previously disclosed, N-desmethylolanzapine which remains in the reaction mixture after completion of the reaction, must be removed from olanzapine. Thus the reaction mixture which is obtained after the methylation of N-desmethylolanzapine can be first extracted with organic solvents, such as ethers, e.g. diethyl ether; esters, e.g. ethyl acetate; or preferably chlorinated organic solvents, such as methylene chloride and chloroform. After separation of the phases, organic phase can be washed with water. An organic acid or substituted organic acid or organic acid derivative of formula RX as previously defined or an organic acid anhydride as previously defined is then added to the organic phase to form a N-substituted N-desmethylolanzapine derivative of formula 2.

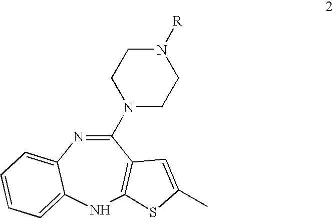

Suitable reagents which can be used for this reaction are organic acids, substituted organic acids and organic acid derivatives such as chloroacetic acid, chloroethylamine, benzyl bromide, phthalic anhydride, acetic anhydride, and the like. For the reaction where said N-substituted N-desmethylolanzapine of formula 2 is formed, different amines, such as dicyclohexylamine, diisopropylamine, triethylamine, diisopropylethylamine, diazabicyclooctane, ethylenediamine, isopropylamine, butylamine, diethylamine, dipropylamine, propylamine, dibutylamine and the like, and different inorganic bases, such as K₂CO₃, Na₂CO₃, NaOH, KOH, LiOH, Ca(OH)₂, NaH, and the like, can be used.

As a final step, olanzapine can optionally be purified through the transformation to an acid addition salt thereof according to the invention as previously described. In this occurrence an organic acid is added to the reaction mixture, such as sulfonic acids or carboxylic acid, preferably oxalic, fumaric or benzoic acid, and the like, more preferably oxalic acid, to form an olanzapine acid addition salt which can be precipitated out of the mixture after cooling and can be filtered off. Aqueous solubility of prepared oxalic acid addition salt of olanzapine is approximately up to about 800 mg/ml.

Olanzapine acid addition salt can be afterwards transformed to pure olanzapine in crystal forms I or II by dissolving olanzapine acid addition salt in water and the pH is adjusted to about 1.0-5.0, preferably to 2.0 by the addition of hydrochloric acid. To the resulting solution charcoal is added. After stirring the mixture for about 5 minutes, charcoal is filtered off and the cake is washed with water. Filtrate and wash water are combined followed by addition of a low boiling organic solvent, such as diethyl ether, methylene chloride, chloroform, ethyl acetate, and the like, preferably methylene chloride. The treatment of the reaction mixture with charcoal at pH of about 2 corresponds to the final purification of olanzapine and allows to get rid of the above described dimer (piperazine 1,4-bis-4-yl-(2-methyl)-10H-thieno-[2,3-b][1,5]benzodiazepine, see scheme 4). Next step is the addition of a base, preferably an inorganic base, such as ammonia, K₂CO₃, KOH, NaH, Na₂CO₃, LiOH, Ca(OH)₂ and the like, more preferably NaOH, to obtain a pH of about 7-11 preferably a pH of about 9-10. After the desired pH is obtained, the mixture can be extracted with a low boiling organic solvent, where different solvents can be used, such as ethers, e.g. diethyl ether; chlorinated hydrocarbons, e.g. methylene chloride, chloroform; esters, e.g. ethyl acetate; and the like, preferably methylene chloride. After the extraction, the organic solvent can be partly removed by rotary evaporation and the residual mixture can be cooled to about −20° C. to about 0° C., preferably about −15° C. to about −5° C. and olanzapine crystal form I is precipitated. Obtained olanzapine contains a very low amount of said dimer (piperazine 1,4-bis-4-yl-(2-methyl)-10H-thieno-[2,3-b][1,5]benzodiazepine), such as lower than about 0.05%

In another embodiment the invention provides a process for the preparation of crystal form II wherein the organic solvent in the very last step is completely removed by rotary evaporation (and not just partly as said in the previous paragraph), followed by the addition of an organic solvent. Different solvents can be used, such as ethers, e.g. diethyl ether; nitriles, e.g. acetonitrile; esters, e.g. ethyl acetate; and the like, preferably diethyl ether, to form a solution of olanzapine from which olanzapine crystal form II can be precipitated after cooling.

Another embodiment of the present invention is a process for producing olanzapine crystal form I via olanzapine acid addition salt. In order to obtain crude olanzapine, the reaction predefined in EP454436B1 was used. The reaction comprises reacting of N-methylpiperazine with 4-amino-2-methyl-10H-thieno[2,3-b][1,5]-benzodiazepine hydrochloride. The reaction is carried out in the presence of a high boiling organic solvent, such as dimethyl sulfoxide, dimethylacetamide, butanol, dimethylformamide, toluene, xylene, ethylbenzene, anisole and the like, preferably dimethyl sulfoxide, at a temperature of about 80-150° C., preferably about 115-130° C.

By the present invention, the resulting solution that contains crude olanzapine after treatment with water is extracted from the obtained solution with an organic solvent whereby different solvents can be used, for example ketones, such as methylisobutylketone; chlorinated hydrocarbons, such as methylene chloride and chloroform, preferably dichloromethane. Thereafter an organic acid, such as carboxylic acid, for example oxalic, fumaric, benzoic acid or sulfonic acids and the like is added to form an acid addition salt of olanzapine. Olanzapine acid addition salt can be filtered off, and optionally dissolved in water and extracted out of the solution with an organic solvent, such as ketones, e.g. methylisobutylketone; chlorinated hydrocarbons, e.g. methylene chloride and chloroform. Afterwards the acid addition salt is isolated by evaporation of solvent. Obtained olanzapine acid addition salt is transformed to olanzapine by first dissolving it in water and the pH is adjusted to about 1.0-5.0, preferably to 2.0 by the addition of hydrochloric acid. To the resulting solution charcoal is added. After stirring the mixture for about 5 minutes, charcoal is filtered off and the cake is washed with water. Filtrate and wash water are combined, followed by the addition of a low boiling organic solvent, such as diethyl ether, methylene chloride, chloroform, ethyl acetate and the like, preferably methylene chloride. Next step is the addition of a base, preferably an inorganic base, such as ammonia, K₂CO₃, KOH, NaH, Na₂CO₃, LiOH, Ca(OH)₂ and the like, more preferably NaOH, to obtain a pH of about 7-11 preferably a pH of about 9-10. After the desired pH is obtained, the mixture can be extracted with a low boiling organic solvent, where different solvents can be used, such as ethers, e.g., diethyl ether; chlorinated hydrocarbons, e.g., methylene chloride, chloroform; esters, e.g. ethyl acetate; and the like, preferably methylene chloride. After the extraction, the organic solvent can be partly removed by rotary evaporation and the residual mixture can be cooled to about −20° C. to about 0° C., preferably to about −15° C. to about −5° C. and olanzapine crystal form I is precipitated and filtered off.

A further optional process consists in the treatment of the remaining solution from any crystallization step of olanzapine in the very final synthesis step. The solution which remains after olanzapine is filtered off, can be directly treated with an organic acid, such as carboxylic acid, for example oxalic, fumaric, benzoic or sulfonic acids and the like. The precipitation of thus formed olanzapine acid addition salt takes place immediately or said remaining solution can be first concentrated by evaporating the solvents and solution can be further diluted by other solvents or mixture of solvents being more suitable for acid addition salt precipitation, preferably by the addition of the mixture of methylene chloride and methanol. Obtained olanzapine acid addition salt can further be transformed to pure olanzapine by the above described procedure.

Olanzapine prepared by processes according to the present invention and excipients may be formulated into pharmaceutical formulations according to methods known in the art. Olanzapine produced by the processes of the present invention is suitable for pharmaceutical use in any pharmaceutical formulation.

Olanzapine produced by the processes of the present invention and formulated accordingly can be then used for the prevention and/or treatment of different mental diseases and conditions such as, for example, disorders of the central nervous system, schizophrenia, hallucination, acute mania, depression and the like.

According to the present invention there is provided a method of treating mental diseases and conditions such as, for example, disorders of the central nervous system, schizophrenia, hallucination, acute mania, depression, and the like which comprises administering a therapeutically effective amount of olanzapine in conjunction with a pharmaceutically acceptable diluent or carrier.

### EXAMPLES

The present invention is illustrated but in no way limited by the following examples:

Abbreviations:

DMAC Dimethylacetamide

DMF Dimethylformamide

DMI 1,3-Dimethyl-2-imidazolidinone

DMPU 1,3-Dimethyl-3,4,5,6-tetrahydro-2(1H)-pyrimidinone

DMSO Dimethyl sulfoxide

NMP N-methylpyrrolidone

Preparation of N-desmethylolanzapin

### Example 1

10.7 g of 4-amino-2-methyl-10H-thieno[2,3-b][1,5]benzodiazepine hydrochloride (0.040 mol) of commercial quality and dark brown colour is suspended in 70 ml of n-butanol and 30 ml of xylene, piperazine (31.5 g; 0.37 mol) is added, heated to reflux and stirred at this temperature for further 4 hours, the end of the reaction is determined by HPLC. After completing the reaction the solvents are evaporated and 200 ml of warm water is added. The formed precipitate is filtered off and washed with 20 ml of ethyl acetate to give 11.0 g of a product (yield: 92%).

### Example 2

10.7 g of 4-amino-2-methyl-10H-thieno[2,3-b][1,5]benzodiazepine hydrochloride (0.040 mol) of commercial quality and dark brown colour is suspended in 100 ml of n-butanol, piperazine (31.5 g; 0.37 mol) is added, heated to reflux and stirred at this temperature for further 8 hours, the end of the reaction is determined by HPLC. After completing the reaction the solvent is evaporated and 200 ml of warm water is added, the formed precipitate is filtered off, washed with 20 ml of ethyl acetate to give 11.7 g of a product (yield: 97%).

### Example 3

10.7 g of 4-amino-2-methyl-10H-thieno[2,3-b][1,5]benzodiazepine hydrochloride (0.040 mol) of commercial quality and dark brown colour is suspended in 50 ml of n-butanol and 50 ml of toluene, piperazine (31.5 g; 0.37 mol) is added, heated to reflux and stirred at this temperature for further 7 hours, the end of the reaction is determined by HPLC. After completing the reaction the solvents are evaporated and 200 ml of warm water is added, the formed precipitate is filtered off, washed with 20 ml of ethyl acetate to give 9.7 g of a product (yield: 81%).

Preparation of Crude Olanzapine from N-desmethylolanzapine

### Example 4

10 g of N-desmethylolanzapine (0.034 mol) is dissolved in 240 ml of tetrahydrofuran and while stirring cooled to −10° C. 9.4 ml of triethylamine and 5 ml (0.080 mol) of methyl iodide are added. The reaction mixture is stirred for 4 hours at −10° C., the end of the reaction is determined by HPLC. After completing the reaction 500 ml of demineralised water is added and tetrahydrofuran is evaporated, the title compound is crystallised as yellow crystals. The precipitate is filtered and washed with water to give 8.6 g of crude olanzapine (yield: 82%).

### Example 5

10 g of N-desmethylolanzapine (0.034 mol) is dissolved in 240 ml of tetrahydrofuran and while stirring cooled to −15° C. 3 g of sodium hydroxide and 5 ml (0.080 mol) of methyl iodide are added. The reaction mixture is stirred for 4 hours at −15° C., the end of the reaction is determined by HPLC. After completing the reaction 500 ml of demineralised water is added and tetrahydrofuran is evaporated, the title compound is crystallised as yellow crystals. The precipitate is filtered off and washed with water to give 10.3 g of crude olanzapine (yield: 98%).

### Example 6

10 g of N-desmethylolanzapine (0.034 mol) is dissolve in 120 ml of tetrahydrofuran and while stirring cooled to −10° C. 1.6 g of sodium hydride and 5 ml (0.080 mol) of methyl iodide are added. The reaction mixture is stirred for 3 hours at −10° C., the end of the reaction is determined by HPLC. After completing the reaction 60 ml of demineralised water is added and tetrahydrofuran is evaporated, then 200 ml of methanol is added, the title compound is crystallised as yellow crystals. The precipitate is filtered off and washed with water to give 10.3 g of crude olanzapine (yield: 98%).

### Example 7

10 g of N-desmethylolanzapine (0.034 mol) is dissolved in 240 ml tetrahydrofuran and while stirring cooled to 0° C. 4.48 g of potassium t-butoxide and 5 ml (0.080 mol) of methyl iodide are added. The reaction mixture is stirred for 2 hours at 0° C., the end of the reaction is determined by HPLC. After completing the reaction 200 ml of demineralised water is added and tetrahydrofuran is evaporated, the title compound is crystallised as yellow crystals. The precipitate is filtered off and washed with water to give 10.4 g of crude olanzapine (yield: 99%).

### Example 8

10 g of N-desmethylolanzapine (0.034 mol) is dissolved in 200 ml of methanol. 20 g of potassium carbonate and 4.4 ml (0.046 mol) of dimethyl sulfate are added. The reaction mixture is stirred for 2 hours at 25° C., the end of the reaction is determined by HPLC. After completing the reaction 350 ml of demineralised water is added and methanol is evaporated, the title compound is crystallised as yellow crystals. The precipitate is filtered off and washed with water to give 6.3 g of crude olanzapine (yield: 60%).

### Example 9

10 g of N-desmethylolanzapine (0.034 mol) is dissolved in 200 ml of acetone and while stirring cooled to −10° C. 20 g of potassium carbonate and 4.4 ml (0.046 mol) of dimethyl sulfate are added. The reaction mixture is stirred for 4 hours at −10° C., the end of the reaction is determined by HPLC. After completing the reaction 350 ml of demineralised water is added and acetone is evaporated, the title compound is crystallised as yellow crystals. The precipitate is filtered off and washed with water to give 4.8 g of crude olanzapine (yield: 46%).

Preparation of Olanzapine Acid Addition Salts from an Isolated Crude Olanzapine

Preparation of Olanzapine Oxalate

### Example 10

To a solution of 0.45 g of olanzapine in 18 ml of DMI, a solution of 0.26 g of oxalic acid in 0.5 ml of DMI is added. After 10 minutes of stirring at 25° C., crystallization begins. The suspension is stirred for one hour at 25° C. and then stirring is continued for one hour on an ice bath. Then the product is isolated by filtration. The product is washed with 25 ml of methylene chloride and dried for two hours at 50° C. in vacuo.

¹H NMR: (300.1 MHz, DMSO-d6)

δ=2.275 (s, 3H, CH₃), 2.625 (s, 4H, NCH₃CO), 2.754 (s. 3H, CH₃), 3.141(4H, piperazinyl-H,), 3.193 (s, 4H, CH₂NCH₃CO) 3.582 (4H, piperazinyl-H), 6.436 (s, 1 H, thiophenyl-H), 6.579 (s, 4H, HC═CH), 6.651 (s, 6.74 (m, 1H, Ar), 6.909 (m, 3H, Ar), 7.954 (s,1H, NH), 9.301 (broad, 3H, NH, OH).

### Example 11

To a solution of 0.45 g of olanzapine in 18 ml of DMI, a solution of 0.26 g of oxalic acid in 0.5 ml of DMAC is added. After 10 minutes of stirring at 25° C., crystallization starts. The suspension is stirred for one hour at 25° C. and then stirring is continued for one hour on an ice bath. Then the product is isolated by filtration. The product is washed with 25 ml of methylene chloride and dried for two hours at 50° C. in vacuo.

| | |
|---|---|
| Yield: | 0.75 g of yellow, crystalline powder |
| mp: | 221° C. |

¹H NMR: (300.1 MHz, DMSO-d6)

δ=1.955 (s, 3H, CH₃CON), (s, 3H, 2.275 (s, 3H, CH₃), 2.625 (s, 4H, NCH₃CO), 2.707 and 2.754 (2 s, 6H, CH₃, CONCH₃), 2.940 (s, 3H, CONCH₃), 3.180 (4H, piperazinyl-H,), 3.583 (4H, piperazinyl-H), 6.416 (s, 1H, thiophenyl-H), 6.416 (m, 1 H, Ar), 6.579 (m, 3H, Ar), 7.846 (s,1H, NH), 8.787 (broad, 3H, NH, OH).

### Example 12

To a suspension of 0.45 g of olanzapine in 18 ml of acetonitrile, a solution of 0.26 g of oxalic acid in 2 ml of acetonitrile is added. The suspension is stirred for one hour at 25° C. and then the stirring is continued for one hour on an ice bath. Then the product is isolated by filtration, washed with 25 ml of acetonitrile and dried for 15 hours at 60° C. in vacuo.

| | |
|---|---|
| Yield: | 0.58 g of yellow, crystalline powder |
| mp: | 235° C. |
| Assay: | 73.5% |
| Oxalic acid: | 24.3% |
| Acetonitrile: | 3 mol % |

### Example 13

To a suspension of 0.45 g of olanzapine in 18 ml of ethanol, a solution of 0.26 g of oxalic acid in 0.5 ml of ethanol is added. After the addition of the solution of oxalic acid, the products start to crystallize from the solution. The suspension is stirred for one hour at 25° C. and then stirring is continued for one hour on an ice bath. Then the product is isolated by filtration. The product is washed with 25 ml of ethanol and dried for two hours at 60° C. in vacuo.

| Yield: | 0.56 g of yellow, crystalline powder |
|---|---|
| mp: | 224° C. |
| Assay: | 75.8% |
| Oxalic acid: | 24.0% |

### Example 14

To a solution of 0.45 g of olanzapine in 18 ml of isopropanol, a solution of 0.26 g of oxalic acid in 2 ml of isopropanol is added. After the addition of the solution of oxalic acid, crystallization starts. The suspension is stirred for one hour at 25° C. and then stirring is continued for one hour on an ice bath. Then the product is isolated by filtration, washed with 25 ml of isopropanol and dried for 15 hours at 60° C. in vacuo.

| | |
|---|---|
| Yield: | 0.60 g of yellow, crystalline powder |
| mp: | 230° C. |
| Assay: | 65.18% |
| Oxalic acid: | 21.5% |
| Isopropanol: | 94 mol % |

Preparation of Olanzapine Fumarate

### Example 15

To a solution of 0.45 g of olanzapine in 18 ml of isopropanol, 0.26 g of fumaric acid is added. The suspension formed is stirred for one hour at 25° C. and then stirring is continued for one hour on an ice bath. Then the product is isolated by filtration. The product is washed with 25 ml of isopropanol and dried for 15 hours at 60° C. in vacuo.

| | |
|---|---|
| Yield: | 0.55 g of yellow, crystalline powder |
| mp: | 231° C. |
| Assay: | 75.5% |
| Fumaric acid: | 17.3% |
| Isopropanol: | 50 mol % |

¹H NMR: (300.1 MHz, DMSO-d6).

δ=2.334 (s, 3H, CH₃), 2.443 (s, 3H, CH₃), 2.729 (4H, piperazinyl-H), 3.434 (4H, piperazinyl-H), 5.753 (s, 1.36H, CH₂Cl₂), 6.346 (s, 1 H, thiophenyl-H), 6.579 (s, 4H, COHC═CHCO), 6.651 (s, 6.643 (m, 1H, ArH), 6.834 (m, 3H, ArH), 7.634 (s,1H, NH).

Preparation of Olanzapine Benzoate

### Example 16

To a solution of 0.45 g of olanzapine in 18 ml of acetone, 0.26 g of benzoic acid is added. The suspension formed is stirred for one hour at 25° C. and then stirring is continued for one hour on an ice bath. Then the product is isolated by filtration. The product is washed with 25 ml of acetone and dried for 15 hours at 60° C. in vacuo.

| | |
|---|---|
| Yield: | 0.60 g of yellow, crystalline powder. |
| mp: | 205° C. |
| Assay: | 70.0% |
| Benzoic acid: | 26.1% |
| Acetone: | 5.2 mol % |

¹H NMR: (300.1 MHz, DMSO-d6)

δ=2.334 (s, 3H, CH₃), 2.443 (s, 3H, CH₃), 2.729 (4H, piperazinyl-H), 3.434 (4H, piperazinyl-H), 6.365 (s, 1H, thiophenyl-H), 6.579 (s, 4H, COHC═CHCO), 6.651 (s, 6.643 (m, 1H, ArH), 6.834 (m, 3H, ArH), 7.686 (s,1H, NH)

Preparation of Olanzapine Acid Addition Salts Directly from the Process of the Synthesis of Olanzapine, Started with the Methylation of N-desmetylolanzapine

Preparation of Olanzapine Oxalate

### Example 17

A solution of 12.0 g of N-desmethylolanzapine (0.040 mol) in a mixture of 180 ml of THF and 120 ml of 1,3-dimethylimidazolinone (DMI) is cooled to approx. −20° C. At −19° C. to the solution, 8.19 g of diisopropylamine and afterwards 13.7 g of methyl iodide (0.097 mol) are added. After stirring the reaction mixture for 45 minutes at −19° C., 6.4 ml of concentrated hydrochloric acid and a solution of 6.36 g of thiourea in 50 ml of water are added and the reaction mixture is stirred for 15 minutes at 20° C.

After the addition of 50 ml of water, the mixture is evaporated at a bath temperature of 35° C. and at 50-60 mbar to a volume of cca. 160 ml. Then 400 ml of water and 120 ml of methylene chloride are added and pH is adjusted to 2.0 with 6 N HCl. After separation of the phases, the water phase is washed twice with 120 ml of methylene chloride. To the water phase, 180 ml of methylene chloride are added and pH is adjusted to 9.0 by the addition of 1 N NaOH. After 5 minutes of stirring, the phases are separated and the alkaline water phase is extracted twice with 90 ml of methylene chloride. The organic phases are combined and the mixture is diluted with 37.5 of methanol and under stirring a solution of 7.46 g of oxalic acid in 10.5 ml of methanol is added within 15 minutes. The resulting suspension is stirred for about 1 hour at approx. 20° C. and afterwards 1 hour at approx. 0° C.

The product is isolated by filtration, washed with 100 ml of methylene chloride and dried for 2 hours at 50° C. in vacuo.

| | |
|---|---|
| Yield: | 15.15 g (69.2%) |
| mp: | 228° C. |
| Assay: | 54.1% |
| HPLC-Purity: | 98.2 area % |
| N-desmethylolanzapine: | 0.95 area % |
| Oxalic acid: | 31.6% |
| DMI: | 6 mol % |
| Methylene chloride: | 0.5 mol % |

### Example 18

A solution of 12.0 g of N-desmethylolanzapine (0.040 mol) in 240 ml of dimethylacetamide (DMAC) is cooled to approx. −20° C. At −20° C. 8.19 g of diisopropylamine are added to the solution and afterwards 7.19 g of methyl iodide (0.050 mol) are added. After stirring the reaction mixture for 95 minutes at −20° C., 6.4 ml of concentrated hydrochloric acid and a solution of 6.36 g of thiourea in 50 ml of water are added and the reaction mixture is stirred for 15 minutes at 20° C. Then 400 ml of water and 120 ml of methylene chloride are added and the pH is adjusted to 2.0 with 6 N HCl. After separation of the phases, the water phase is washed twice with 140 ml of methylene chloride. To the water phase, 180 ml of methylene chloride are added and pH is adjusted to 9.0 by the addition of 1 N NaOH. After 5 minutes of stirring, the phases are separated and the alkaline water phase is extracted twice with 90 ml of methylene chloride. The organic phases are combined and 380 mg of acetic anhydride is added and the mixture is stirred for 5 minutes. Then the mixture is diluted with 37.5 of methanol and under stirring, a solution of 7.46 g of oxalic acid in 10.5 ml of methanol is added within 15 minutes. The resulting suspension is stirred for about 1 hour at approx. 20° C. and afterwards 1 hour at approx. 0° C. The product is isolated by filtration, washed with 100 ml of methylene chloride and dried for 15 hours at 25° C. in vacuo.

| | |
|---|---|
| Yield: | 13.76 g (72.0%) |
| mp: | 233° C. |
| Assay: | 59.4% |
| HPLC-Purity: | 98.3 area % |
| N-desmethylolanzapine: | 0.15 area % |
| Oxalic acid: | 29.1% |
| Methylene chloride: | 69.9 mol % |
| DMAC: | 2.3 mol % |

### Example 19

A solution of 12.0 g of N-desmethylolanzapine (0.040 mol) in 240 ml of 1,3-dimethyl-3,4,5,6-tetrahydro-1,3-pyrimidinone is cooled to approx. −20° C. At −20° C. 8.19 g of diisopropylamine are added to the solution and afterwards 7.57 g of methyl iodide (0.053 mol) are added. After stirring the reaction mixture for 60 minutes at −20° C., 7.2 ml of concentrated hydrochloric acid and a solution of 6.36 g of thiourea in 50 ml of water are added and the reaction mixture is heated to 20° C. and stirred for 5 minutes at the same temperature. Then 400 ml of water and 120 ml of methylene chloride are added and pH is adjusted to 2.0 with 6 N HCl. After separation of the layers, the water layer is washed twice with 120 ml of methylene chloride. To the water phase 180 ml of methylene chloride are added and pH is adjusted to 9.0 by the addition of 1 N NaOH. After 5 minutes of stirring, the layers are separated and the alkaline water layer is extracted twice with 90 ml of methylene chloride. The organic layers are combined and 380 mg of acetic anhydride are added and the mixture is stirred for 5 minutes. Then the solvent is evaporated in vacuo and the oily residue is dissolved in a mixture of 360 ml of methylene chloride, 37.5 ml of methanol and 0.72 ml of water. To this solution seeds of olanzapine oxalate are added and while stirring a solution of 7.71 g of oxalic acid in 10.5 ml of methanol within 20 minutes is added. The resulting suspension is stirred for about 1 hour at approx. 25° C. and afterwards 1 hour at approx. 0° C. The product is isolated by filtration, washed with 100 ml of methylene chloride and dried for 15 hours at 60° C. in vacuo.

| | |
|---|---|
| Yield: | 14.8 g (82.4%) of yellow, crystalline powder |
| mp: | 229° C. |
| Assay: | 64.1% |
| HPLC-Purity: | 99.5 area % |
| N-desmethylolanzapine: | <0.1 area % |
| Oxalic acid: | 32.4% |
| Methylene chloride: | 10.4 mol % (drying 24 h at 50° C.) |
| DMPU: | 0.5 mol % |

### Example 20

A mixture of 30.0 g of 4-amino-2-methyl-10H-thieno[2,3-b][1,5]-benzodiazepine hydrochloride (0.113 mol) and 81 ml of N-methylpiperazine (0.729 mol) in 186 ml of DMSO is heated to 117° C. After 17 hours of stirring and bubbling nitrogen through the mixture at this temperature, the resulting solution is cooled to room temperature (R.T.) and then 570 ml of methylene chloride and 570 ml of water are added. After stirring the mixture for 5 minutes, the layers are separated. The alkaline water layer is extracted with 300 ml of methylene chloride. To the combined organic layers, 250 ml of water are added and pH is adjusted to 2.0 by the addition of 6 M HCl. After separating the layers, the organic layer is extracted twice with 90 ml of water. The combined acidic water layers are treated with 4.5 g of charcoal. After 5 minutes of stirring, charcoal is filtered off and the cake is washed with 100 ml of water. Filtrate and wash water are combined and after adding of 950 ml of methylene chloride, pH is adjusted to 9.0 by the addition of 5 M NaOH. After separating the layers, the alkaline water layer is extracted with 125 ml of methylene chloride. The organic layers are combined and evaporated in vacuo. The oily residue is dissolved in a mixture of 1075 ml of methylene chloride, 140 ml of methanol and 3.6 ml of water and heated to about 29-30° C. After adding seeds of olanzapine oxalate to the solution, a solution of 18.7 g of oxalic acid in 27 ml of methanol is added within 30 minutes. The resulting suspension is stirred for about 1 hour at approx. 25° C. and afterwards 2 hours at approx. 0° C. The product is isolated by filtration, washed with 150 ml of methylene chloride and dried for 6 hours at 60° C. in vacuo.

| | |
|---|---|
| Yield: | 43.3 g (82.2%) of yellow, crystalline powder |
| mp: | 224° C. |
| Assay: | 62.7% |
| HPLC-Purity: | 99.6 area % |
| Oxalic acid: | 26.1% |
| Methylene chloride: | 22 mol % |

Preparation of Olanzapine Fumarate

### Example 21

To the solution of olanzapine (obtained from 12.0 g of starting N-desmethylolanzapine, according to example 16) in the mixture of 360 ml of methylene chloride, 37.5 of methanol and 0.72 mg of water, seeds of olanzapine fumarate crystal and 0.96 g of fumaric acid are added. The resulting suspension is stirred for about 1 hour at 25° C. and afterwards 2 hours at approx. 0° C. The product is isolated by filtration, washed with 150 ml of methylene chloride and dried for 6 hours at 60° C. in vacuo.

| | |
|---|---|
| Yield: | 11.4 g (65.7%) of light yellow, crystalline |
| | powder |
| mp: | 217° C. |
| Assay: | 65.7% |
| HPLC-Purity: | 97.8 area % |
| N-desmethylolanzapine: | 0.15 area % |
| Fumaric acid: | 23.2% |
| Methylene chloride: | 48 mol % |

### Example 22

A solution of olanzapine prepared from 30.0 g of 4-amino-2-methyl-10H-thieno[2,3-b][1,5]-benozodiazepine hydrochloride (0.113 mol) and 81 ml of N-methylpiperazine (0.729 mol) in 186 ml of DMSO according to example 5, is heated to 29-30° C. At this temperature, seeds of olanzapine fumarate and 14.4 g of fumaric acid are added. The resulting suspension is stirred for about 1 hour at 29-30° C. and afterwards 2 hours at approx. 0° C. The product is isolated by filtration, washed with 150 ml of methylene chloride and dried for 6 hours at 60° C. in vacuo.

| | |
|---|---|
| Yield: | 41.9 g (85.2%) of light yellow, crystalline powder |
| mp: | 217° C. |
| Assay: | 68.5% |
| HPLC-Purity: | 99.7 area % |
| Fumaric acid: | 23.0% |
| Methylene chloride: | 48 mol % |

Preparation of Pure Olanzapine Crystal Form I from Olanzapine Oxalate

### Example 23

7.40 g of olanzapine oxalate are dissolved in 75 ml of water and the pH of the solution is adjusted to 2.0 by the addition of 6 N HCl. To the resulting clear solution of olanzapine oxalate, 0.75 g of charcoal is added. After stirring for 5 minutes, charcoal is filtered off and the cake is washed with 50 ml of water. Filtrate and wash water are combined and after the addition of 125 ml of methylene chloride, pH of combined mixture is adjusted to 9.0 by the addition of 1 N NaOH. After stirring for 5 minutes, the layers are separated and the water phase is extracted with 25 ml of methylene chloride. The organic layers are combined and after drying with sodium carbonate, the solution is concentrated in vacuo to a volume of 27 ml. Then the concentrated solution is heated to the reflux temperature at a normal pressure and after adding seeds of olanzapine crystal form I, the solution is immediately cooled on an ice bath. Adding said seeds is continued until olanzapine begins to crystallize. The resulting suspension is stirred for 15 minutes on an ice bath and then for 15 minutes at about −20° C. Then olanzapine is isolated by filtration. The cake is washed with 3 ml of methylene chloride having a temperature of −20° C. The product is dried for two days at 25° C. in vacuo.

| Yield: | 3.63 g (72.6%) |
|---|---|
| HPLC-Purity: | 99.9% |

IR—identical with olanzapine crystal form I reference substance

XRD—identical with olanzapine crystal form I reference substance

Preparation of Pure Olanzapine Crystal Form II from Olanzapine Oxalate

### Example 24

7.40 g of olanzapine oxalate are dissolved in 75 ml of water and pH of the solution is adjusted to 2.0 by the addition of 6 N HCl. To the resulting clear solution of olanzapine oxalate, 0.75 g of charcoal is added. After stirring for 5 minutes, charcoal is filtered off and the cake is washed with 50 ml of water.

Filtrate and wash water are combined and after the addition of 125 ml of methylene chloride, pH of combined mixture is adjusted to 8-10 by the addition of 1 N NaOH. After stirring for 5 minutes, the layers are separated and the water phase is extracted with 25 ml of methylene chloride. The organic layers are combined and the methylene chloride is evaporated. Then ethyl acetate is added and olanzapine starts to crystallize. The resulting suspension is stirred for 15 minutes on an ice bath. Then olanzapine is isolated by filtration. The product is dried for two hours at 60° C. in vacuo.

| Yield: | 3.4 g |
|---|---|
| HPLC-Purity: | 99.9% |

IR—identical with olanzapine crystal form II reference substance

XRD—identical with olanzapine crystal form II reference substance

Preparation of Pure Olanzapine from N-desmethylolanzapine Via an Acid Addition Salt of Olanzapine

### Example 25

A solution of 20.0 g of N-desmethylolanzapine (0.067 mol) in a mixture of 150 ml of THF and 60 ml of DMAC is cooled to approx. −15° C. At −15° C. to the reaction mixture, 20 ml of diisopropylamine are added and afterwards 6 ml of methyl iodide (0.96 mol) in 30 ml of THF are added within 30-40 minutes. After stirring the reaction mixture for another 60 minutes at −5 to −10° C., 16 ml of concentrated hydrochloric acid in 100 ml of water and a solution of 3.3 g of thiourea in 100 ml of water are added and the reaction mixture is stirred for 15 minutes at 20° C.

The THF is evaporated at a bath temperature of 35° C. and at a pressure of 50-60 mbar to a volume of cca. 200 ml. Then 300 ml of methylene chloride are added and pH is adjusted to 8.5-9 with 40% NaOH. After separation of the phases, the water phase is washed twice with 100 ml of methylene chloride. Organic phases are combined and washed five times with 100 ml of water. The organic phases are combined, 0.5 ml of acetic anhydride is added and the mixture is stirred for 5 minutes. A solution of 10.34 g of oxalic acid dihydrate in 40 ml of methanol is added within 15 minutes. The resulting suspension is stirred for about 1 hour at approx. 20° C. and afterwards 1 hour at approx. 0° C. The product is isolated by filtration, washed with 100 ml of methylene chloride and dried for 2 hours at 50° C. in vacuo.

Yield: 25.1 g.

25 g of olanzapine oxalate are dissolved in 250 ml of water and pH of the solution is adjusted to 2.0 by the addition of 6 N HCl. To the resulting clear solution of olanzapine oxalate, 2.5 g of charcoal is added. After stirring for 5 minutes, charcoal is filtered off and the cake is washed with 50 ml of water. Filtrate and wash water are combined and after the addition of 300 ml of methylene chloride, pH is adjusted to 9-10 by the addition of 10 N NaOH. After stirring for 5 minutes, the layers are separated and the water phase is extracted with 50 ml of methylene chloride. The organic layers are combined and the solution is concentrated in vacuo to a volume of 50 ml. Then the concentrated solution is heated to reflux temperature at a normal pressure and after adding seeds of olanzapine crystal form I, the solution is immediately cooled on an ice bath. Adding said seeds is continued until olanzapine starts to crystallize. The resulting suspension is stirred for 15 minutes on an ice bath and then for 15 minutes at −20° C. Then olanzapine is isolated by filtration. The cake is washed with 10 ml of methylene chloride having a temperature of −20° C. The product is dried for four hours at 80° C. in vacuo.

Yield: 11.5 g

Table 1 shows the analytical results of the intermediate olanzapine oxalate and the final product olanzapine prepared according to the process described in Example 16.

TABLE 1

| | olanzapine oxalate | olanzapine |
|---|---|---|
| HPLC-purity | 98.3% | 99.8% |
| m.p. | 228° C. | 191° C. |
| N-desmethylolanzapine | ≦0.10% | ≦0.05% |
| N,N-dimethylolanzapine | ≦1.0% | ≦0.05% |
| acetylolanzapine | ≦0.20% | ≦0.05% |
| piperazine 1,4-bis-4-yl-(2- | ≦0.6% | ≦0.05% |
| methyl)-10H-thieno-[2,3- | | |
| b][1,5]benzodiazepine | | |
| (dimer) | | |

Description of the HPLC Analysis:

HPLC was carried out in Waters Alliance 2695 separations module, detector PDA 2996, software Empower 5.0. Buffer is 15 mM NaH₂PO₄, pH=6.2: 2.34 g NaH₂PO₄×H₂O/1000 ml water, pH with 5 M NaOH to 6.2

Chromatographic conditions:

1. Mobile phase:
A: Buffer 15 mM NaH₂PO₄, pH=6.2/ACN/MeOH 70/20/10 (v/v/v)
B: Buffer 15 mM NaH₂PO₄, pH=6.2/MeOH 25/75 (v/v)

2. Column: BetaBasic C-8 3 μm, 100×4.6 mm

3. Temperature: 30° C.

4. Flow rate: 0.55 ml/min

5. Wavelength: 254 nm

6. Injection volume: 5 l

7. Gradient table:

| t | % A | % B |
|---|---|---|
| 0 | 83 | 17 |
| 12 | 83 | 17 |
| 25 | 0 | 100 |
| 45 | 0 | 100 |
| 46 | 83 | 17 |

Description of the ¹H NMR Analysis:

The ¹H spectra were recorded on a Bruker Avance 300 instrument using standard instrumental procedures. Samples were dissolved in DMSO-d6 at a concentration of approximately 15 mg/ml and measured at ambient temperature. The solvent signal was used as internal reference: 2.50 ppm for ¹H NMR. The operating frequency was 300 MHz for ¹H.

1. A process for the purification of olanzapine characterized in that said process comprises the following steps:
a) mixing olanzapine with an organic acid in an organic solvent or a mixture of organic solvents to form an olanzapine acid addition salt,
b) precipitating and isolating the olanzopine acid addition salt and,
c) transformation of the olanzapine acid addition salt to olanzapine.

2. The process according to claim 1 wherein the organic acid in step (a) is selected from the group consisting of sulfonic acids or carboxylic acid.

3. The process according to claim 2 wherein the carboxylic acid is selected from the group consisting of fumaric acid and benzoic acid.

4. The process according to claim 1 wherein the organic solvent in step (a) is selected from the group consisting of tetrahydrofuran, acetone, dimethylformamide and acetonitrile.

5. The process according to claim 1 wherein the mixture of organic solvents in step (a) is a mixture of tetrahydrofuran with at least one polar solvent.

6. The process according to claim 5 wherein said polar solvent is selected from the group consisting of dimethylformamide, dimethylacetamide, N-methylpyrrolidone, 1,3-dimethyl-3,4,5,6-tetrahydro-2(1H)-pyrimidinone, 1,3-imethyl-2-imidazolidinone, tetramethylurea, dimethyl sulfoxide, sulfolane, acetone and acetonitrile.

7. The process according to claim 1 characterized in that step (c) comprises the following substeps:
i) dissolving an acid addition salt of olanzapine in water,
ii) adjusting pH of the obtained solution to about 8-10,
iii) extracting olanzapine from the water phase to the organic solvent phase and
iv) isolating the acid addition salt of olanzapine from the organic solvent phase by concentrating the solution and separation of the crystals.

8-21. (canceled)

22. A process for the preparation of olanzapine in the form of an acid addition salt characterized in that said process comprises the following steps:
a) 4-amino-2-methyl-10H-thieno[2,3-b][1,5]benzodiazepine hydrochloride is reacted with N-methylpiperazine to yield olanzapine and
b) the obtained olanzapine is transformed to an acid addition salt thereof.

23. The process according to claim 22 characterized in that step (b) comprises the following substeps:
i) the obtained reaction mixture is diluted with water,
ii) the diluted reaction mixture is extracted with an organic solvent,
iii) the organic phase is evaporated and the residue is diluted with a second solvent to obtain a solution,
iv) an organic acid is added to the solution to precipitate olanzapine acid addition salt and
v) precipitated olanzapine acid addition salt is isolated by separation of crystals.

24. A process for the preparation of olanzapine in the form of an acid addition salt characterized in that said process comprises the following steps:
a) N-desmethylolanzapine is reacted with a methylating agent to yield olanzapine,
b) the obtained reaction mixture is diluted with water and acidified with an acid,
c) to the reaction mixture, an organic solvent is added and the phases are separated,
d) the obtained water phase is neutralized and olanzapine is extracted with an organic solvent to obtain the organic solvent phase and
e) an organic acid or substituted organic acid or an organic acid derivative of formula RX; wherein R represents an organic radical such as acetyl, propionyl, chloroacetyl and X is selected from a group of Cl, Br or I; or an organic acid anhydride; is added to the organic phase to form a N substituted N-desmethylolanzopine derivative of formula 2 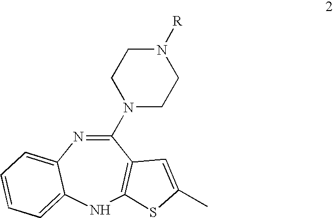
f) the obtained organic solvent phase is optionally evaporated and the residue is diluted with a second organic solvent,
g) an organic acid is added either to the obtained diluted solution or directly to the olanzapine extract from said extraction in step (d) and
h) precipitated olanzapine acid addition salt is isolated by separation of the crystals.

25. The process according to claim 24 wherein the organic solvent in steps (c) and (d) is a chlorinated solvent.

26. The process according to claim 25 wherein said chlorinated solvent is methylene chloride.

27. The process according to claim 24 wherein the organic solvent in steps (c) and (d) is methylene chloride and said second solvent in step (f) is methanol.

28-34. (canceled)

35. Olanzapine prepared according to the processes disclosed in claim 1 characterized in that N-desmethylolanzapine content in the final product of olanzapine is less than 0.1%.

36. Olanzapine prepared according to the processes disclosed in claim 1 that contains less than 0.05% of piperazine 1,4-bis-4-yl-(2-methyl)-10H-thieno-[2,3-b][1,5]benzodiazepine.

37-43. (canceled)
